(19) United States
(12) Patent Application Publication
(10) Pub. No.: US 2015/0320591 A1
(43) Pub. Date: Nov. 12, 2015

(54) Systems and Methods for In-Ear Fluid Vestibular and/or Cranial Nerve Caloric Stimulation

(71) Applicant: Scion NeuroStim, LLC, Raleigh, NC (US)

(72) Inventors: Lanty L. Smith, Raleigh, NC (US); Lesco L. Rogers, Raleigh, NC (US); Robert D. Black, Chapel Hill, NC (US)

(21) Appl. No.: 14/707,268

(22) Filed: May 8, 2015

Related U.S. Application Data
(60) Provisional application No. 61/990,867, filed on May 9, 2014.

(51) Int. Cl. A61F 7/12; A61B 6/03; A61B 8/08; A61B 5/0484; A61B 8/06; A61B 5/04; A61B 5/00

(52) U.S. Cl. CPC A61F 7/12; A61B 5/04009; A61B 6/037; A61B 5/0059; A61B 5/0484; A61B 8/06; A61B 8/488; A61F 2007/0005

(57) ABSTRACT

An in-ear stimulation system for administering caloric stimulation to the ear canal of a subject includes (a) a conduit configured to deliver a fluid to calorically stimulate an ear canal of a subject; and (b) a fluid control unit comprising a controller configured to control a flow of the fluid to the conduit and/or to control a caloric profile of the fluid such that a temperature of the fluid changes through time when the fluid is being delivered to the ear canal via the conduit.

### RELATED APPLICATION

This application claims priority from U.S. Provisional Application No. 61/990,867, filed May 9, 2014, the disclosure of which is hereby incorporated herein in its entirety.

### FIELD OF THE INVENTION

The present invention relates to systems and methods for delivering stimulation to the nervous system and/or the vestibular system of an individual, and in particular, for in-ear fluid vestibular and/or cranial nerve caloric stimulation delivered to the external auditory canal.

### BACKGROUND

Caloric vestibular stimulation (“CVS”) has long been known as a diagnostic procedure for testing the function of the vestibular system. In the traditional hospital setting, water caloric tests are used to assess levels of consciousness during acute or chronic brain injury. The brain injury may be due to head trauma or a central nervous system event such as a stroke. Other brain injuries occur in the presence of metabolic abnormalities (e.g., kidney disease, diabetes), seizures, or toxic levels of controlled substances or alcohol. CVS may be used to evaluate the integrity of the vestibular organs of each ear. Patients with balance difficulties are often evaluated with CVS. Conventional diagnostic CVS systems typically deliver water or a gas at a constant temperature to the ear.

U.S. Patent Publication No. 2003/0195588 to Fischell et al. discusses a stimulator in an ear canal that is adapted to provide magnetic, electrical, audible, tactile or caloric stimulation. Fischell proposes a ring-shaped caloric transducer strip on an ear canal sensor/stimulator system that may result in relatively slow thermal changes of the ear canal.

U.S. Patent Publication Nos. 2011/0313499 and 2011/0313498 to Rogers et al. disclose in-ear stimulators for administering thermal stimulation to the ear canal of a subject having an earpiece with a thermoelectric device thermally coupled to the earpiece to stimulate the vestibular and/or cranial nerve. Relatively fast temperature changes may be achieved using the thermoelectric device.

Accordingly, systems and associated methods useful for delivering stimulation to the nervous system and/or the vestibular system of an individual that may be capable of relatively fast temperature changes are potentially beneficial to take full advantage of physiological responses that are useful in diagnosing and/or treating a variety of medical conditions.

### SUMMARY OF EMBODIMENTS OF THE INVENTION

An in-ear stimulation system for administering caloric stimulation to the ear canal of a subject includes (a) a conduit configured to deliver a fluid to calorically stimulate an ear canal of a subject; and (b) a fluid control unit comprising a controller configured to control a flow of the fluid to the conduit and/or to control a caloric profile of the fluid such that a temperature of the fluid changes through time when the fluid is being delivered to the ear canal via the conduit. In some embodiments, a second conduit that is configured to deliver the fluid to another ear canal of the subject is provided so that the fluid control unit may control the caloric profile to both ear canals of the subject independently.

### BRIEF DESCRIPTION OF THE DRAWINGS

The accompanying drawings, which are incorporated in and constitute a part of the specification, illustrate embodiments of the invention and, together with the description, serve to explain principles of the invention.

FIGS. 1-6 and 7A-7B are schematic diagrams of in-ear stimulation systems for administering caloric stimulation to the ear canal of a subject using a fluid controller and a conduit having an earpiece according to some embodiments.

FIG. 8 is a schematic diagram of an in-ear stimulation system for administering caloric stimulation to the ear canal of a subject using a fluid controller and a conduit having a fluid delivery system around the ear according to some embodiments.

FIG. 9 is a cross sectional view of the fluid delivery system of FIG. 8.

FIG. 10 is a schematic diagram of an in-ear stimulation system for administering caloric stimulation to the ear canal of a subject using a fluid controller and a conduit having a Joule-Thomson expansion chamber.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

### Definitions

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, infrared, or semiconductor system, apparatus, or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory such as an SD card), an optical fiber, and a portable compact disc read-only memory (CD-ROM).

As used herein, the term “vestibular system” has the meaning ascribed to it in the medical arts and includes but is not limited to those portions of the inner ear known as the vestibular apparatus and the vestibulocochlear nerve. The vestibular system, therefore, further includes, but is not limited to, those parts of the brain that process signals from the vestibulocochlear nerve.

“Treatment,” “treat,” and “treating” refer to reversing, alleviating, reducing the severity of, delaying the onset of, inhibiting the progress of, or preventing a disease or disorder as described herein, or at least one symptom of a disease or disorder as described herein (e.g., treating one or more of tremors, bradykinesia, rigidity or postural instability associated with Parkinson's disease; treating one or more of intrusive symptoms (e.g., dissociative states, flashbacks, intrusive emotions, intrusive memories, nightmares, and night terrors), avoidant symptoms (e.g., avoiding emotions, avoiding relationships, avoiding responsibility for others, avoiding situations reminiscent of the traumatic event), hyperarousal symptoms (e.g., exaggerated startle reaction, explosive outbursts, extreme vigilance, irritability, panic symptoms, sleep disturbance) associated with post-traumatic stress disorder). In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved—for example, to prevent or delay their recurrence. Treatment may comprise providing neuroprotection, enhancing cognition and/or increasing cognitive reserve. Treatment may be as an adjuvant treatment as further described herein.

“Adjuvant treatment” as described herein refers to a treatment session in which the delivery of one or more thermal waveforms to the vestibular system and/or the nervous system of a patient modifies the effect(s) of one or more active agents and/or therapies. For example, the delivery of one or more thermal waveforms to the vestibular system and/or the nervous system of a patient may enhance the effectiveness of a pharmaceutical agent (by restoring the therapeutic efficacy of a drug to which the patient had previously become habituated, for example). Likewise, the delivery of one or more thermal waveforms via a caloric profile to the vestibular system and/or the nervous system of a patient may enhance the effectiveness of counseling or psychotherapy. In some embodiments, delivery of one or more thermal waveforms to the vestibular system and/or the nervous system of a patient may reduce or eliminate the need for one or more active agents and/or therapies. Adjuvant treatments may be effectuated by delivering one or more thermal waveforms to the vestibular system and/or the nervous system of a patient prior to, currently with and/or after administration of one or more active agents and/or therapies.

“Chronic treatment,” “Chronically treating,” or the like refers to a therapeutic treatment carried out at least 2 to 3 times a week (or in some embodiments at least daily) over an extended period of time (typically at least one to two weeks, and in some embodiments at least one to two months), for as long as required to achieve and/or maintain therapeutic efficacy for the particular condition or disorder for which the treatment is carried out.

“Waveform” or “waveform stimulus” or “caloric profile” as used herein refers to the thermal stimulus (heating, cooling) delivered to the ear canal of a subject through a suitable apparatus to carry out the methods described herein. “Waveform” is not to be confused with “frequency,” the latter term concerning the rate of delivery of a particular waveform. The term “waveform” is used herein to refer to one complete cycle thereof, unless additional cycles (of the same, or different, waveform) are indicated. As discussed further below, time-varying waveforms may be preferred over constant temperature applications in carrying out the present invention.

“Actively controlled waveform” or “actively controlled time-varying waveform” as used herein refers to a waveform stimulus in which the intensity of the stimulus or temperature of the earpiece delivering that stimulus, is repeatedly adjusted, or substantially continuously adjusted or driven, throughout the treatment session, typically by control circuitry or a controller in response to active feedback from a suitably situated temperature sensor (e.g., a temperature sensor mounted on the earpiece being driven by a thermoelectric device), so that drift of the thermal stimulus from that which is intended for delivery which would otherwise occur due to patient contact is minimized

In general, a waveform stimulus used to carry out the present invention comprises a leading edge, a peak, and a trailing edge. If a first waveform stimulus is followed by a second waveform stimulus, then the minimal stimulus point therebetween is referred to as a trough.

The first waveform of a treatment session is initiated at a start point, which start point may be the at or about the subject's body temperature at the time the treatment session is initiated (typically a range of about 34 to 38 degrees Centigrade, around a normal body temperature of about 37 degrees Centigrade. The lower point, 34, is due to the coolness of the ear canal. It typically will not be above about 37 unless the patient is febrile). Note that, while the subject's ear canal may be slightly less than body temperature (e.g., about 34 to 36 degrees Centigrade), the starting temperature for the waveform is typically body temperature (the temp of the inner ear), or about 37 degrees Centigrade. In some embodiments, however, the temperature of the treatment device may not have equilibrated with the ear canal prior to the start of the treatment session, and in such case the start point for at least the first waveform stimulus may be at a value closer to room temperature (about 23 to 26 degrees Centigrade).

The waveform leading edge is preferably ramped or time-varying: that is, the amplitude of the waveform increases through a plurality of different temperature points over time (e.g., at least 5, 10, or 15 or more distinct temperature points, and in some embodiments at least 50, 100, or 150 or more distinct temperature points, from start to peak). The shape of the leading edge may be a linear ramp, a curved ramp (e.g., convex or concave; logarithmic or exponential), or a combination thereof. A vertical cut may be included in the waveform leading edge, so long as the remaining portion of the leading edge progresses through a plurality of different temperature points over time as noted above.

The peak of the waveform represents the amplitude of the waveform as compared to the subject's body temperature. In general, an amplitude of at least 5 or 7 degrees Centigrade is preferred for both heating and cooling waveform stimulation. In general, an amplitude of up to 20 degrees Centigrade is preferred for cooling waveform stimulation. In general, an amplitude of up to 8 or 10 degrees Centigrade is preferred for heating waveform stimulus. The peak of the waveform may be truncated (that is, the waveform may reach an extended temperature plateau), so long as the desired characteristics of the leading edge, and preferably trailing edge, are retained. For heating waveforms, truncated peaks of long duration (that is, maximum heat for a long duration) are less preferred, particularly at higher heats, due to potential burning sensation. In some embodiments, the temperature applied in the ear canal is between about 13° C. and 43° C. The temperature applied in the ear canal range from about 22-24° C. below body temperature to about 6-10° C. above body temperature.

The waveform trailing edge is preferably ramped or time-varying: that is, the amplitude of the waveform decreases through a plurality of different temperature points over time (e.g., at least 5, 10, or 15 or more distinct temperature points, or in some embodiments at least 50, 100, or 150 or more distinct temperature points, from peak to trough). The shape of the trailing edge may be a linear ramp, a curved ramp (e.g., convex or concave; logarithmic or exponential), or a combination thereof. A vertical cut may again be included in the waveform trailing edge, so long as the remaining portion of the trailing edge progresses through a plurality of different temperature points over time as noted above.

The duration of the waveform stimulus (or the frequency of that waveform stimulus) is the time from the onset of the leading edge to either the conclusion of the trailing edge or (in the case of a vertically cut waveform followed by a subsequent waveform). In general, each waveform stimulus has a duration, or frequency, of from one or two minutes up to ten or twenty minutes.

A treatment session may have a total duration of five or ten minutes, up to 20 or 40 minutes or more, depending on factors such as the specific waveform or waveforms delivered, the patient, the condition being treated, etc. For example, in some embodiments a treatment session may be 60 minutes or more. In some embodiments, treatment sessions may include breaks between stimulation, such as breaks of a minute or more.

In a treatment session, a plurality of waveforms may be delivered in sequence. In general, a treatment session will comprise 1, 2 or 3 waveforms, up to about 10 or 20 or more waveforms delivered sequentially. Each individual waveform may be the same, or different, from the other. When a waveform is followed by a subsequent waveform, the minimum stimulus point (minimum heating or cooling) between is referred to as the trough. Like a peak, the trough may be truncated, so long as the desired characteristics of the trailing edge, and the following next leading edge, are retained. While the trough may represent a return to the subject's current body temperature, in some embodiments minor thermal stimulation (cooling or heating; e.g., by 1 or 2 degrees up to 4 or 5 degrees Centigrade) may continue to be applied at the trough (or through a truncated trough).

Treatment sessions are preferably once a day, though in some embodiments more frequent treatment sessions (e.g. two or three times a day) may be employed. Day-to-day treatments may be by any suitable schedule: every day; every other day; twice a week; as needed by the subject, etc. The overall pattern of treatment is thus typically chronic (in contrast to “acute,” as used in one-time experimental studies).

Subjects may be treated with the present invention for any reason. In some embodiments, disorders for which treatment may be carried out include, include, but are not limited to, migraine headaches (acute and chronic), depression, anxiety (e.g. as experienced in post-traumatic stress disorder (“PTSD”) or other anxiety disorders), spatial neglect, Parkinson's disease, seizures (e.g., epileptic seizures), diabetes (e.g., type I and type II diabetes), etc.

Headaches that may be treated by the methods and apparatuses of the present invention include, but are not limited to, primary headaches (e.g., migraine headaches, tension-type headaches, trigeminal autonomic cephalagias and other primary headaches, such as cluster headaches, cough headaches and exertional headaches) and secondary headaches. See, e.g., International Headache Society Classification ICHD-II.

Migraine headaches that may be treated by the methods and apparatuses of the present invention may be acute/chronic and unilateral/bilateral. The migraine headache may be of any type, including, but not limited to, migraine with aura, migraine without aura, hemiplegic migraine, opthalmoplegic migraine, retinal migraine, basilar artery migraine, abdominal migraine, vestibular migraine and probable migraine. As used herein, the term “vesibular migraine” refers to migraine with associated vestibular symptoms, including, but not limited to, head motion intolerance, unsteadiness, dizziness and vertigo. Vestibular migraine includes, but is not limited to, those conditions sometimes referred to as vertigo with migraine, migraine-associated dizziness, migraine-related vestibulopathy, migrainous vertigo and migraine-related vertigo. See, e.g., Teggi et al., HEADACHE 49:435-444 (2009).

Tension-type headaches that may be treated by the methods and apparatuses of the present invention, include, but are not limited to, infrequent episodic tension-type headaches, frequent episodic tension-type headaches, chronic tension-type headache and probable tension-type headache.

Trigeminal autonomic cephalagias that may be treated by the methods and apparatuses of the present invention, include, but are not limited to, cluster headaches, paroxysmal hemicranias, short-lasting unilateral neuralgiform headache attacks with conjunctival injection and tearing and probable trigeminal autonomic cephalagias. Cluster headache, sometimes referred to as “suicide headache,” is considered different from migraine headache. Cluster headache is a neurological disease that involves, as its most prominent feature, an immense degree of pain. “Cluster” refers to the tendency of these headaches to occur periodically, with active periods interrupted by spontaneous remissions. The cause of the disease is currently unknown. Cluster headaches affect approximately 0.1% of the population, and men are more commonly affected than women (in contrast to migraine headache, where women are more commonly affected than men).

Other primary headaches that may be treated by the methods and apparatuses of the present invention, include, but are not limited to, primary cough headache, primary exertional headache, primary headache associated with sexual activity, hypnic headache, primary thunderclap headache, hemicranias continua and new daily-persistent headache.

Additional disorders and conditions that can be treated by the methods and systems of the present invention include, but are not limited to, neuropathic pain (e.g., migraine headaches), tinnitus, brain injury (acute brain injury, excitotoxic brain injury, traumatic brain injury, etc.), spinal cord injury, body image or integrity disorders (e.g., spatial neglect), visual intrusive imagery, neuropsychiatric disorders (e.g. depression), bipolar disorder, neurodegenerative disorders (e.g. Parkinson's disease), asthma, dementia, insomnia, stroke (including post-stroke aphasia), cellular ischemia, metabolic disorders, (e.g., diabetes), post-traumatic stress disorder (“PTSD”), addictive disorders, sensory disorders, motor disorders, and cognitive disorders.

Sensory disorders that may be treated by the methods and apparatuses of the present invention include, but are not limited to, vertigo, dizziness, seasickness, travel sickness cybersickness, sensory processing disorder, hyperacusis, fibromyalgia, neuropathic pain (including, but not limited to, complex regional pain syndrome, phantom limb pain, thalamic pain syndrome, craniofacial pain, cranial neuropathy, autonomic neuropathy, and peripheral neuropathy (including, but not limited to, entrapment-, heredity-, acute inflammatory-, diabetes-, alcoholism-, industrial toxin-, Leprosy-, Epstein Barr Virus-, liver disease-, ischemia-, and drug-induced neuropathy)), numbness, hemianesthesia, and nerve/root plexus disorders (including, but not limited to, traumatic radiculopathies, neoplastic radiculopathies, vaculitis, and radiation plexopathy).

Motor disorders that may be treated by the method and apparatuses of the present invention include, but are not limited to, upper motor neuron disorders such as spastic paraplegia, lower motor neuron disorders such as spinal muscular atrophy and bulbar palsy, combined upper and lower motor neuron syndromes such as familial amyotrophic lateral sclerosis and primary lateral sclerosis, and movement disorders (including, but not limited to, Parkinson's disease, tremor, dystonia, Tourette Syndrome, myoclonus, chorea, nystagmus, spasticity, agraphia, dysgraphia, alien limb syndrome, and drug-induced movement disorders).

Cognitive disorders that may be treated by the method and apparatuses of the present invention include, but are not limited to, schizophrenia, addiction, anxiety disorders, depression, bipolar disorder, dementia, insomnia, narcolepsy, autism, Alzheimer's disease, anomia, aphasia, dysphasia, parosmia, spatial neglect, attention deficit hyperactivity disorder, obsessive compulsive disorder, eating disorders, body image disorders, body integrity disorders, post-traumatic stress disorder, intrusive imagery disorders, and mutism.

Metabolic disorders that may be treated by the present invention include type I and type II diabetes, hypertension, obesity, etc.

Addiction, addictive disorders, or addictive behavior that may be treated by the present invention includes, but is not limited to, alcohol addiction, tobacco or nicotine addiction (e.g., using the present invention as a smoking cessation aid), drug addictions (e.g., opiates, oxycontin, amphetamines, etc.), food addictions (compulsive eating disorders), etc.

In some embodiments, the subject has two or more of the above conditions, and both conditions are treated concurrently with the methods and systems of the invention. For example, a subject with both depression and anxiety (e.g., PTSD) can be treated for both, concurrently, with the methods and systems of the present invention.

The methods and systems according to embodiments of the present invention utilize a temperature-controlled fluid to induce physiological and/or psychological responses in a subject for medically diagnostic and/or therapeutic purposes. Subjects to be treated and/or stimulated with the methods, devices and systems of the present invention include both human subjects and animal subjects. In particular, embodiments of the present invention may be used to diagnose and/or treat mammalian subjects such as cats, dogs, monkeys, etc. for medical research or veterinary purposes.

As noted above, embodiments according to the present invention utilize a temperature-controlled fluid to provide an in-ear stimulator for administering thermal stimulation in the ear canal of the subject. The ear canal serves as a useful conduit to the individual's vestibular system and to the vestibulocochlear nerve. Without wishing to be bound by any particular theory, it is believed that thermal stimulation of the vestibular system is translated into electrical stimulation within the central nervous system (“CNS”) and propagated throughout the brain, including but not limited to the brain stem, resulting in certain physiological changes that may be useful in treating various disease states (increased blood flow, generation of neurotransmitters, etc). See, e.g., Zhang, et al. Chinese Medical I. 121:12:1120 (2008) (demonstrating increased ascorbic acid concentration in response to cold water CVS).

### System

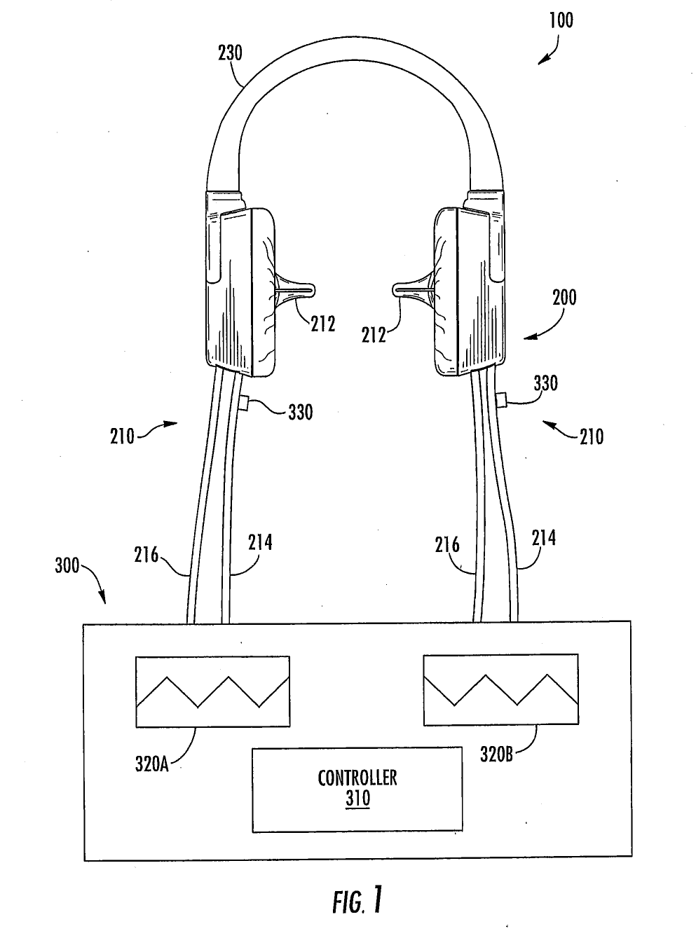

As shown in FIG. 1, an in-ear stimulation system 100 includes a delivery apparatus 200 and a fluid control unit 300. The delivery apparatus 200 includes a conduit 210 that is configured to deliver a fluid to calorically stimulate an ear canal of a subject. As illustrated, the conduit 210 is held in position on a subject by a headset 230. The fluid control unit 300 includes a controller 310 that controls a flow of the fluid to the conduit 210 and/or controls a caloric profile of the fluid in the conduit 210. In some embodiments, the controller 310 controls the caloric profile of the fluid so that a temperature of the fluid changes through time according to a desired caloric profile protocol when the fluid is being delivered to the ear canal via the conduit 210. The conduit 210 includes an earpiece 212, an inlet 214 that delivers fluid to the earpiece 212, and an outlet 214 that returns the fluid to the control unit 300 for reuse or disposal. As illustrated, the apparatus 200 includes two conduits 210 and two earpieces 212 for delivering a caloric stimulation to both ear canals of the subject. It should be understood, however, that either single ear or dual ear stimulation may be used. The earpieces 212 may be formed of a thermally conductive material, and the fluid may contact the earpieces 212 and/or flow into an internal cavity of the earpieces 212 to change a temperature of the ear canal. The caloric profile of the fluid may include a waveform as described herein for changing a thermal stimulus delivered to the ear canal.

As illustrated in FIG. 1, the controller 310 includes two caloric profile generators 320A, 320B and temperature sensors 330 for controlling the caloric profile of the fluid delivered through the two conduits 210, respectively. The temperature sensors 330 detect a temperature of the fluid or in a region adjacent the fluid in the conduit 210 and communicates the detected temperature to the control unit 300. Accordingly, the temperature sensors 330 provide a temperature feedback to the control unit 300 so that the controller 310 may increase or decrease the heating/cooling of the fluid in response to an actual temperature sensed by the sensors 330 to provide a desired temperature of the fluid in the conduit 210 and/or to account for thermal changes of the fluid in the conduit 210. Although the temperature sensors 330 are positioned adjacent the inlets 214, it should be understood that the sensors 330 may be positioned at any suitable position, including on or adjacent the earpiece 212 or the outlets 216.

As illustrated, two different caloric profiles may be delivered via the conduits 210 to the ear canals of the subject to provide different temperatures in each ear at the same time. For example, the caloric profiles of the fluid in the conduits 210 may be out-of-phase with each other such that a similarly shaped temperature gradient is delivered to the ear canals at different times that are out-of-phase with one another. In some embodiments, the caloric profiles may be selected such that when a slope of the first caloric profile is increasing, a slope of the second caloric profile decreases, and when a slope of the first caloric profile is decreasing, a slope of the second caloric profile increases. As another example, the caloric profiles may be configured to deliver heating and cooling profiles that are different in each ear, e.g., so that when the first caloric profile cools one of the subject's ear canals (i.e., delivers a fluid with a temperature that is lower than the subject's body temperature), the second caloric profile heats the other of the subject's ear canals (i.e., delivers a fluid with a temperature that is higher than the subject's body temperature). Various time-varying caloric waveforms may be used as discussed in U.S. Patent Publication Nos. 2011/0313499 and 2011/0313498 to Rogers et al., the disclosures of which are hereby incorporated by reference in their entireties. In some embodiments, the first and second caloric profiles are configured to increase and/or decrease a temperature at the first and second conduits at a rate of about 15° C. per minute or more. In some embodiments, faster temperature changes (slew rates) may be achieved with a temperature-controlled fluid, such as water, as compared to the TED devices of U.S. Patent Publication Nos. 2011/0313499 and 2011/0313498 to Rogers et al. In some embodiments, slew rates of 20° C.-100° C., 30° C.-60° C. or 100° C. per minute or more may be achieved.

In some embodiments, the vestibular stimulation of a subject may be maintained over an extended period of time. In contrast, constant temperature CVS systems typically used for diagnostics generally stimulate the vestibular system for a short duration. In some embodiments, the first and second caloric profiles are configured to maintain a vestibular stimulation of the subject for at least five minutes. The stimulation of the vestibular system may be sufficient to be confirmed by various techniques, such as functional imaging of the brain stem. Functional imaging may include Electroencephalography (EEG), Magnetoencephalography (MEG), functional Magnetic Resonance Imaging (fMRI), Positron Emission Tomography (PET) and/or Optical Imaging. Vestibular stimulation using time-varying CVS may also be observed by secondary effects, including cerebral blood flow velocity oscillations, heart rate modulation (generally resulting in a lower heart rate, changes in breathing rate, and/or changes in pupil diameters. Heart rate modulation may be measured, for example, with an ECG to determine beat-to-beat intervals, which may be used to calculate various cardiac parameters, e.g., in frequency and/or time. Respiratory rates may be measured in various ways, including a strain gauge that is wrapped around the chest. Breathing rates may change during CVS and may be measured by autonomic tone. Pupillometry may also be used to measure changes in pupil diameters as a result of CVS. In particular embodiments, vestibular stimulation may cause cerebral blood flow velocity oscillations that may be observed using transcranial Doppler sonography. In particular embodiments, the vestibular stimulation may be sufficient to alter a vestibular phasic firing rate to thereby induce nystagmus over a period of at least five minutes, and the nystagmus may be sufficient to be detected using videonystagmography and/or electronystagmography.

### Fluid Controllers

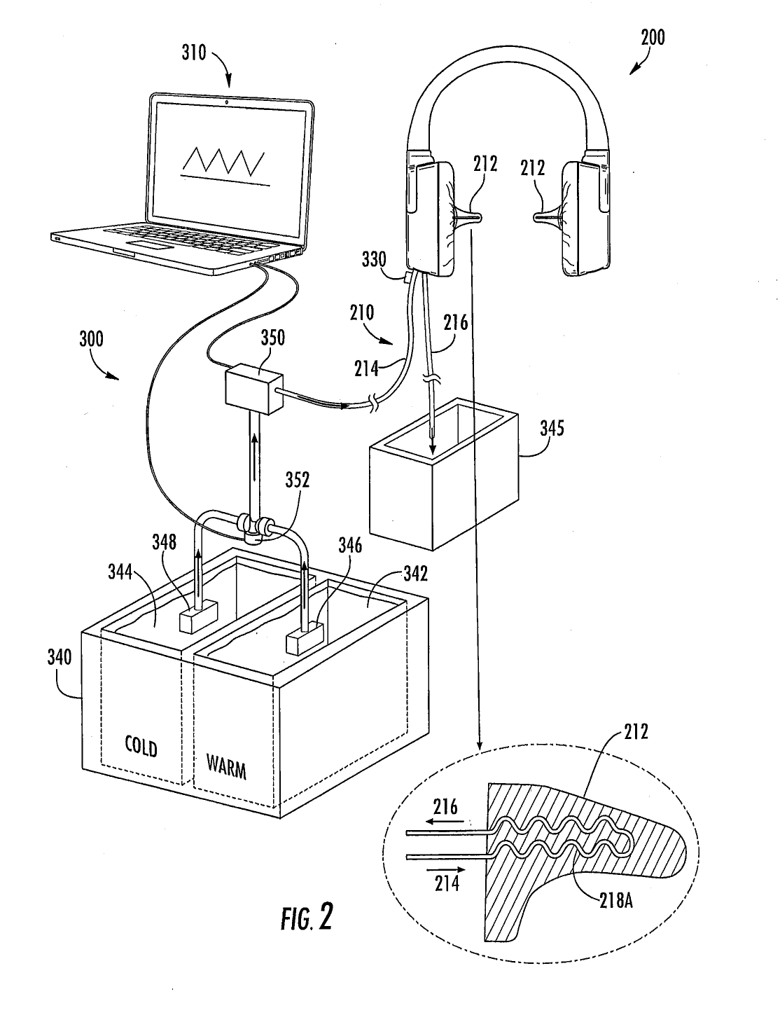

Any suitable techniques for controlling a temperature of the fluid delivered via the conduit 210 may be used. For example, as shown in FIGS. 2, 4, and 8, the control unit 300 includes a reservoir container 340 with a warm reservoir 342 and a cold reservoir 344. Each reservoir 342, 344 includes a respective pump 346, 348 for pumping the fluid from the reservoirs 342, 344 to the conduit 210. A mixer 350 and/or flow controller 352 control how much warm water and how much cold water is obtained from the reservoirs 342, 344 in order to obtain the desired temperature and/or change in temperature over time. Examples of flow controllers include Model U802 Ultra-High-Purity (UHP) flow controllers from McMillan Products (Georgetown, Tex., USA). After the fluid passes through the inlet 214, earpiece 212 and outlet 216, the fluid exits into a waste container 345. In some embodiments, the fluid in the waste container 345 may be recycled into the reservoir container 340, e.g., after the fluid is heated and/or cooled to an appropriate temperature.

In some embodiments, the warm reservoir 342 may be heated to a predetermined temperature by a heater, such as a water heater, and the cold reservoir 344 may be maintained at a predetermined temperature by a cooler, such as a refrigeration system. Any suitable fluid may be used, including liquids (e.g., water, low viscosity oils, alcohol or ammonia) or a gas. The pumps 346, 348 may be a submersible pump or a peristaltic pump or other suitable fluid pumping configuration.

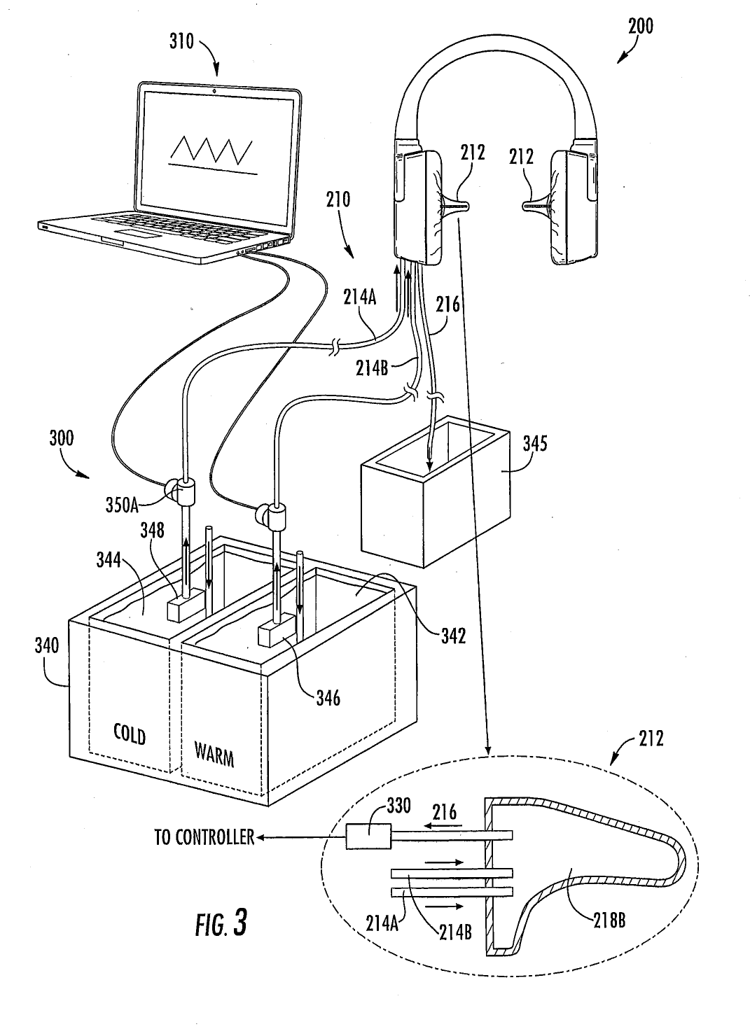

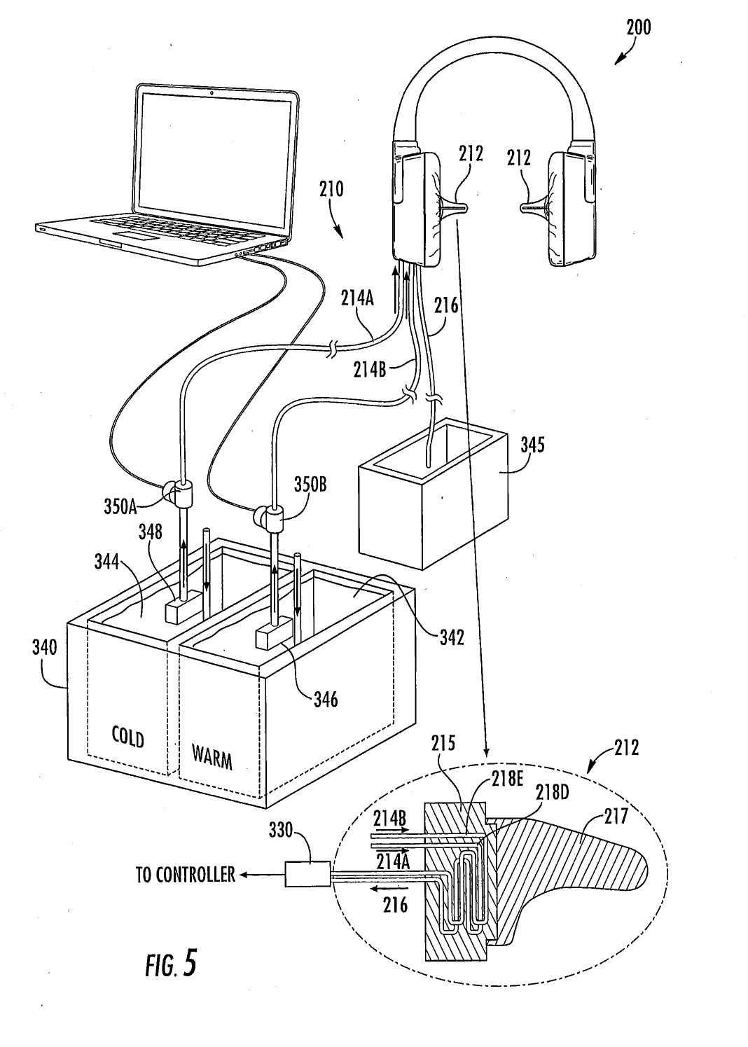

As illustrated in FIGS. 3 and 5, the conduit 210 may include separate inlets 214A, 214B for cold fluid (inlet 214A) and warm fluid (inlet 214B). The flow of fluid from the warm water reservoir 342 and the cold water reservoir 344 may be controlled by fluid flow controllers 350A, 350B, respectively. The temperature sensor 330 on the outlet 216 provides a feedback temperature to the controller 310 so that the controller 310 can modify the flow rates of the flow controllers 350A, 350B to achieve a desired caloric profile. That is, if the temperature at the sensor 330 is too high, then the controller 310 may increase the flow rate from the controller 350A for the cold water reservoir 344 and/or decrease the flow rate from the controller 350B for the warm water reservoir 342. If the temperature at the sensor 330 is too low, then the controller 310 may decrease the flow rate from the controller 350A for the cold water reservoir 344 and/or increase the flow rate from the controller 350B for the warm water reservoir 342. In some embodiments, the total flow from the controllers 350A, 350B may be maintained at a constant flow rate and the proportions of warm/cold may be adjusted by the controllers 350A, 350B to achieve a predetermined temperature and/or temperature gradient.

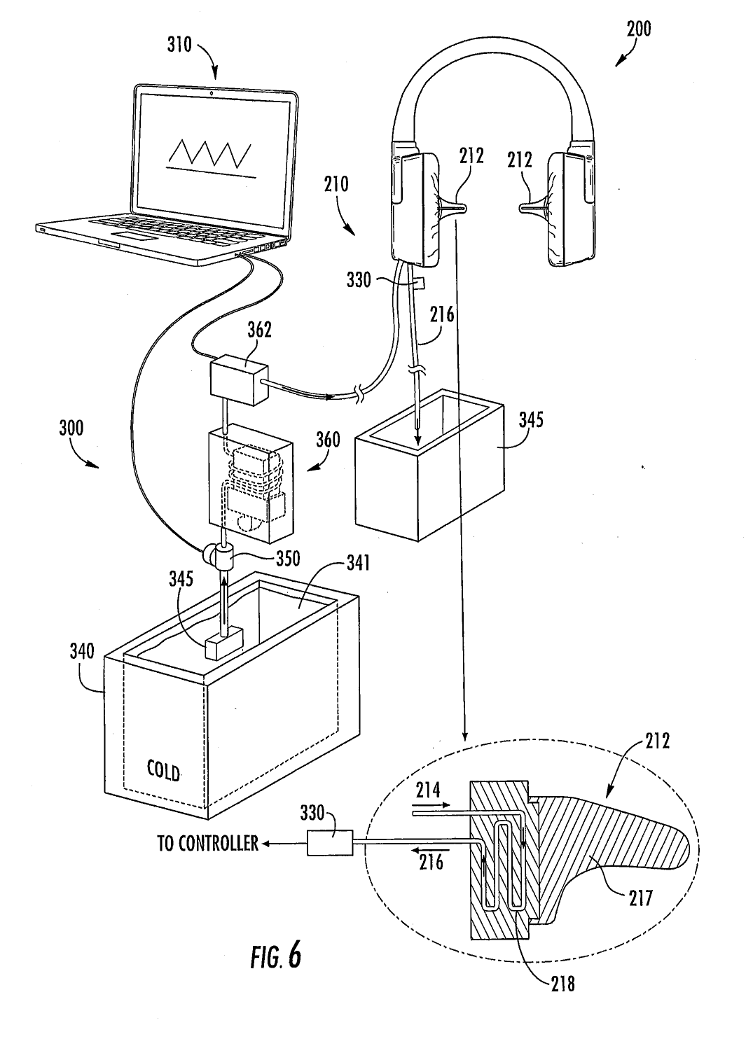

As shown in FIG. 6, a single cold fluid source or reservoir 341 may be used. The reservoir 341 may be connected by a flow controller 350 to a heat exchanger 360 that is controlled by a heat controller 362. The heat exchanger 360 heats the fluid, for example, using a resistive wire, gas heat source or other heat sources known to those of skill in the art. The amount of heat and/or the flow rate of the fluid may be controlled by the controllers 350 and 362 to vary the temperature of the fluid flowing into the inlet 214 according to the desired caloric profile.

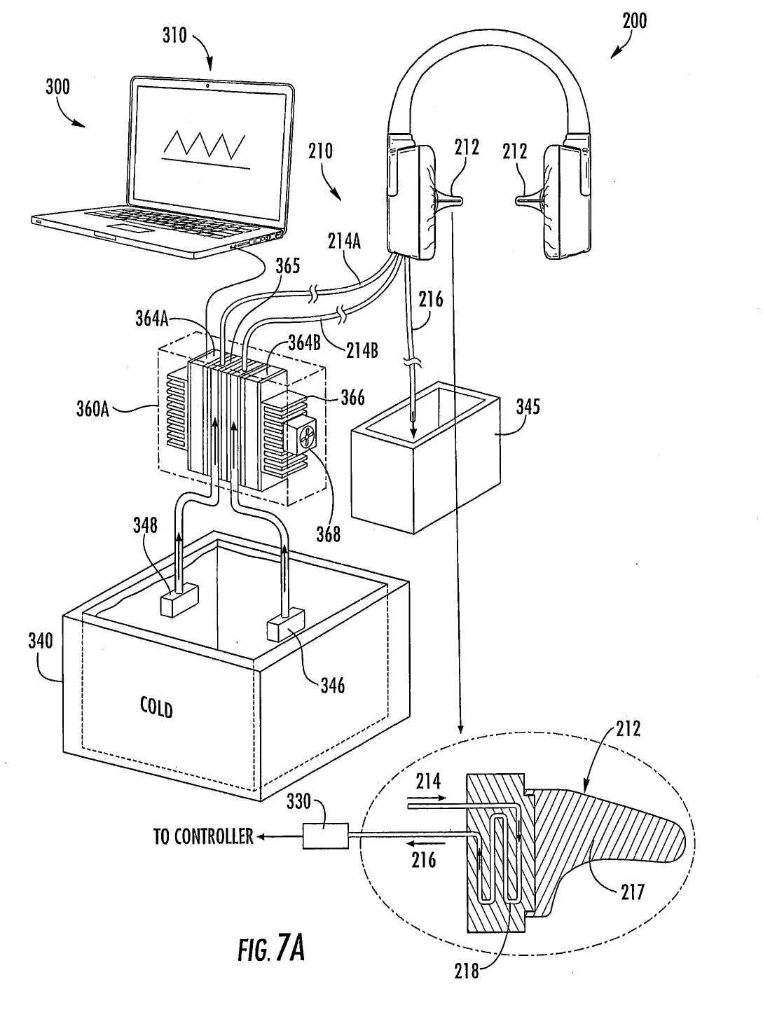

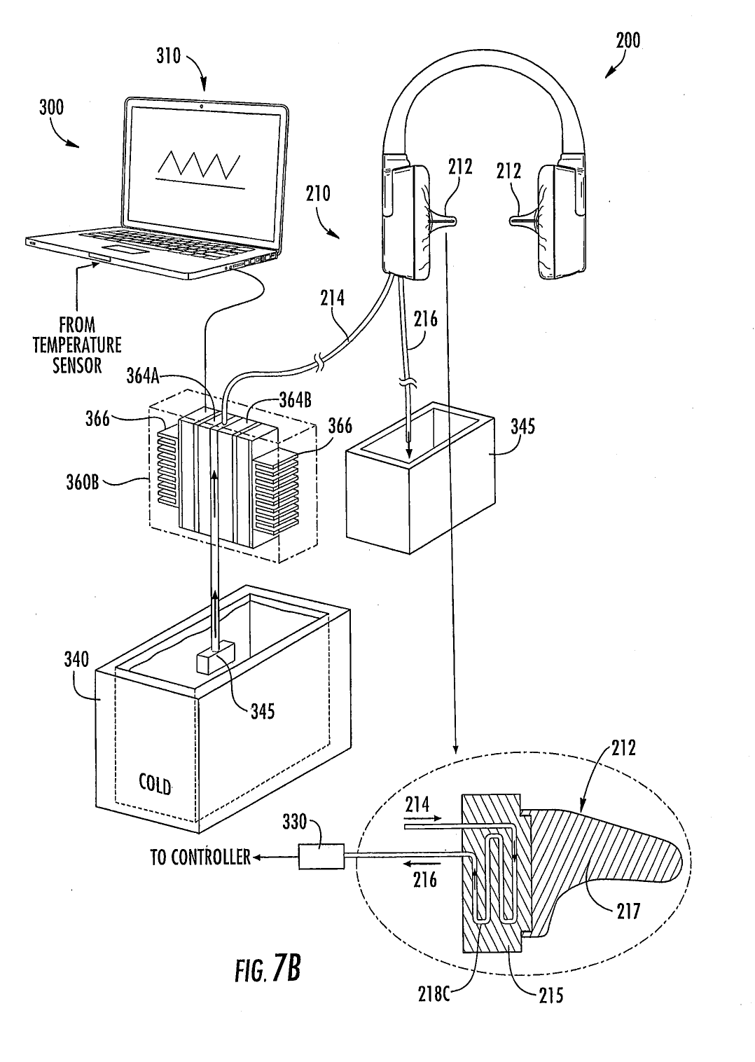

As shown in FIG. 7A, the heat exchanger 360A may include separate heating/cooling elements for the inlets 214A, 214B. As illustrated, Peltier or thermoelectric devices (TEDs) 364A, 364B are positioned adjacent the respective inlets 214A, 214B to heat and/or cool the fluid to a predetermined caloric profile. Heat sinks 366 and cooling fans 368 may also be provided. The fluid flow or amount of warm/cold fluid from the inlets 214A, 214B and/or the degree to which the fluid is heated and/or cooled by the heat exchanger 360 may be controlled to provide the desired temperature and to change the temperature in the earpiece 212 over time.

As shown in FIG. 7B, the heat exchanger 360B may be used with a single inlet 214. The heat exchanger 360B may include a warm side Peltier or TED 364A and a cold side Peltier or TED 364B for heating/cooling the fluid in the inlet 214 to a desired temperature and to change the temperature over time. Although a cold fluid reservoir 345 is illustrated, it should be understood that any suitable fluid source may be used, including a running water from a standard tap water supply.

Although various fluid controllers are illustrated in FIGS. 2-8, it should be understood that any suitable fluid controller, including temperature and fluid flow controllers, are within the scope of the invention. Moreover, although the fluid controllers in FIGS. 2-8 are shown as heating/cooling one fluid conduit or earpiece, it should be understood that the fluid controllers may be used to heat/cool fluid to two fluid conduits or earpieces for dual-ear thermal stimulation. Additionally, any suitable fluid conduit, including the various fluid conduits described herein with respect to FIGS. 2-9 may be used with any of the fluid controllers interchangeably.

### Fluid Conduits

Any suitable fluid conduit may be used to provide caloric stimulation to the ear canal. For example, as shown in FIG. 2, the conduit 210 includes an earpiece 212 that may include a cavity such as a pathway 218A in the earpiece 212 for containing the heating/cooling fluid therein. In some embodiments, the earpiece 212 may be a three-dimensional printed earpiece, which may be custom-manufactured and shaped for an individual subject's ear canal, e.g., based on a molding or scanned image of the ear canal.

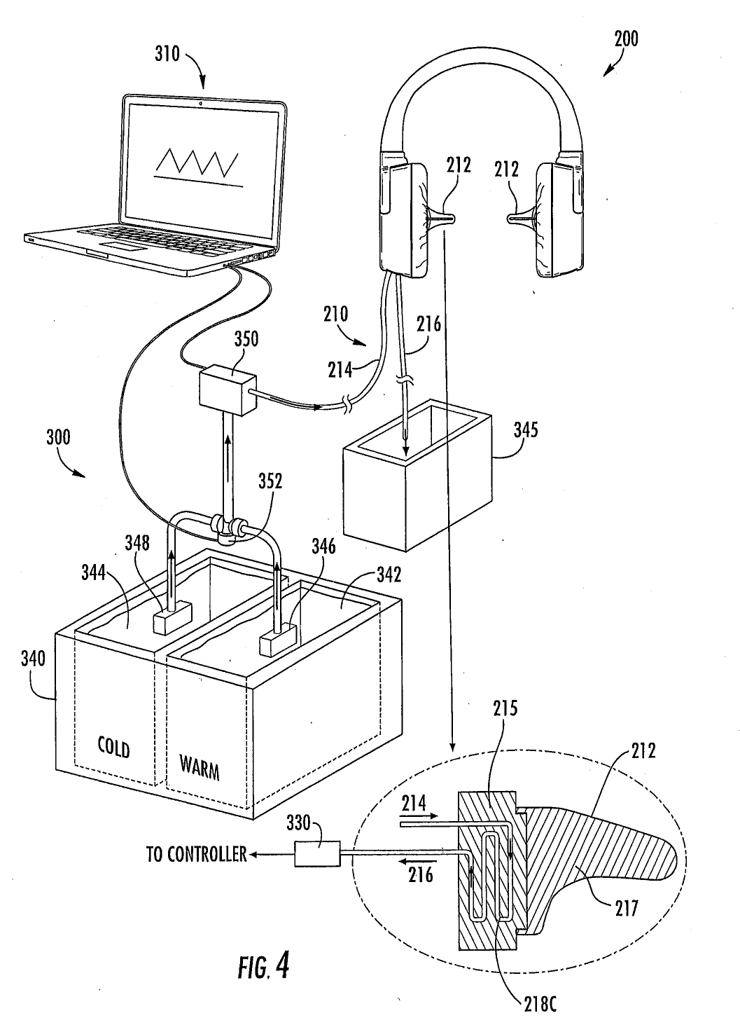

Any suitable earpiece may be used. For example, as illustrated in FIG. 3, the earpiece 212 includes a hollow cavity 218B for receiving the fluid therein. As shown in FIGS. 4, 6, 7A and 7B, the earpiece 212 includes a heat exchange block 215 having a passageway 218C that is connected to a solid earpiece 217. As shown in FIG. 5, the heat exchange block 215 includes two passageways 218D, 218E for warm and cold fluid inlets, respectively. Moreover, in some embodiments, the two passageways 218D, 218E may be formed through the earpiece 217 and/or the heat exchange block 215 may be omitted. It should be understood that the earpiece 212 may be formed of thermally conductive materials, including, but not limited to aluminum, copper, stainless steel, brass, titanium, nickel and/or alloys thereof. In some embodiments polymers may be used (including elastomeric polymers). In some embodiments, a material with lower thermal conductivity may be used provided that the thickness of the material (e.g., the wall of the earpiece) is sufficiently thin so as to allow sufficient thermal conductivity. The earpiece 212 may be formed of rigid, semi-rigid or flexible materials. The earpiece 212 may be formed of a flexible material that is conformable to the ear canal during use or the earpiece 212 may be formed of rigid or semi-rigid materials that are shaped so as to form a snug fit in the ear canal, including custom-manufactured and/or three-dimensionally printed earpieces.

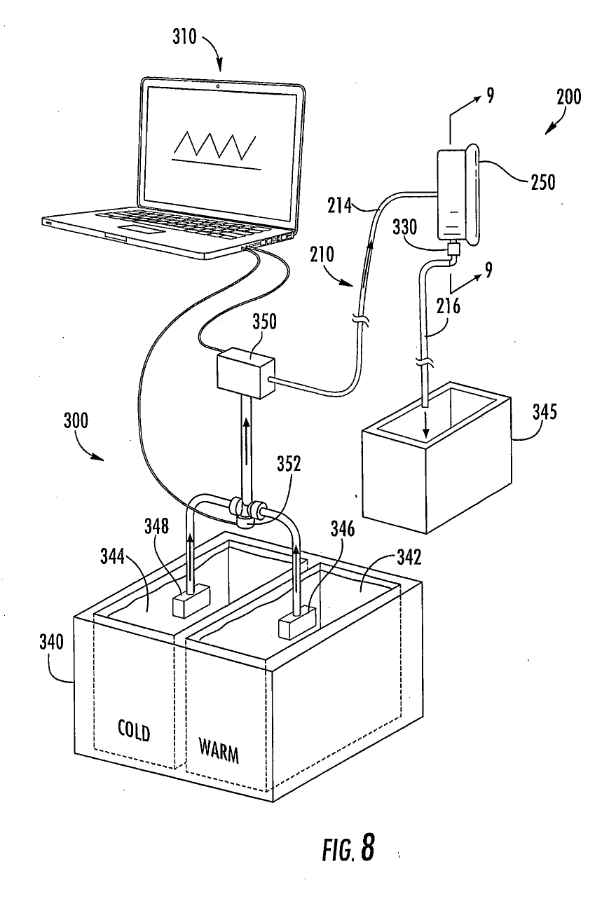

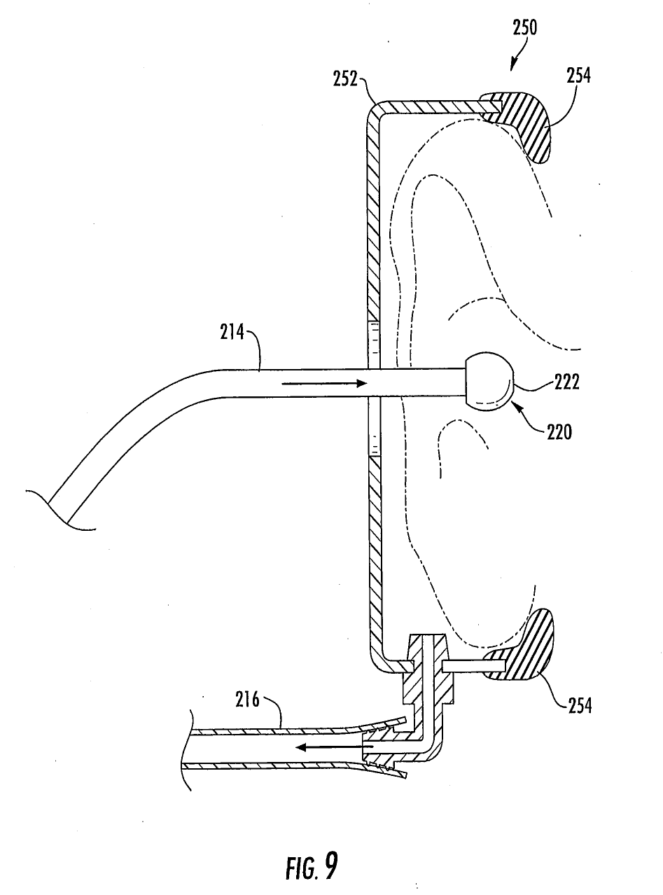

In some embodiments, the earpiece 212 may be omitted, and in some embodiments, the fluid may be in direct contact with the ear canal. As shown in FIGS. 8-9, the fluid conduit 210 includes a fluid delivery device 250 that is connected to the inlet 214 and outlet 216. The inlet 214 includes a knob 220 that has an aperture 222 therein that releases fluid into the ear canal as shown in FIG. 9. The device 250 includes an outer housing 252 and a sealing member 254 for sealing and containing the fluid adjacent the ear. Accordingly, fluid enters the ear canal via the aperture 222 and flows out of the ear canal into the outlet 216.

Although the fluid control system 300 illustrated in FIG. 8 is shown with respect to two reservoirs 346, 348 and a fluid mixer/controller 350, any suitable fluid controller may be used to deliver a fluid having a desired caloric profile to provide thermal stimulation of the ear canal. Water may be used as the fluid in the open system of FIGS. 8-9, which permits direct contact between the fluid and the ear canal, and fluids that may irritate the subject's ear or require cleaning after treatment may be avoided.

Any suitable fluid flow rates may be used. For example, in closed-flow systems as shown in FIGS. 1-7, flow rates of about 500-1000 ml per minute may be used for a liquid. In an open system as shown in FIGS. 8-9 in which fluid (e.g., water) directly contacts the ear canal, flow rates of about 300 ml-600 ml of fluid (e.g., water) may be used.

### Joule-Thomson System

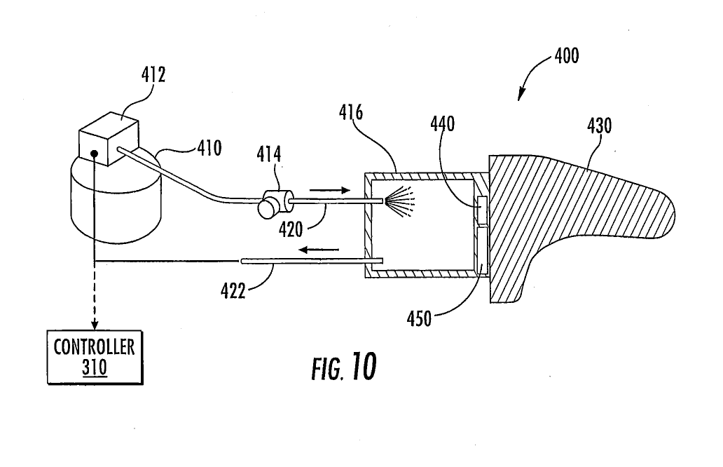

In some embodiments, a Joule-Thomson expansion chamber may be used to control a temperature of an earpiece. As illustrated in FIG. 10, a controller 310 is connected to a thermal delivery system 400. The thermal delivery system 400 includes a fluid supply 410 and fluid flow controllers 412, 414, which are connected to a fluid inlet 420 of a chamber 416. The chamber 416 includes an outlet 420, a temperature sensor 440 and a heater 450 (such as a resistive heater or TED) and is thermally connected to an earpiece 430. In this configuration, a fluid, such as a gas, may be supplied to the expansion chamber 416, where it expands and cools according to the Joule-Thomson effect. Accordingly, the expansion chamber 416 cools the earpiece 430 while the heater 450 warms the earpiece 430. By alternating the heating and cooling effects of the expansion chamber 416 and the heater 450, a time-varying caloric profile may be delivered to the ear canal for caloric stimulation.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An in-ear stimulation system for administering caloric stimulation to the ear canal of a subject, the system comprising:
(a) a conduit configured to deliver a fluid to calorically stimulate an ear canal of a subject; and
(b) a fluid control unit comprising a controller configured to control a flow of the fluid to the conduit and/or to control a caloric profile of the fluid such that a temperature of the fluid changes through time when the fluid is being delivered to the ear canal via the conduit.

2. The in-ear stimulation system of claim 1, wherein the conduit is a first conduit, the in-ear stimulation system further comprising a second conduit configured to deliver the fluid to another ear canal of the subject.

3. The in-ear stimulation system of claim 2, wherein the caloric profile comprises a first caloric profile, and the controller of the fluid control unit is configured to control the first caloric profile of the fluid delivered to the first conduit and to control a second caloric profile of the fluid delivered to the second conduit, wherein the first caloric profile in the first conduit is different than the second caloric profile in the second conduit.

4. The in-ear stimulation system of claim 3, wherein the first caloric profile is out-of-phase with the second caloric profile.

5. The in-ear stimulation system of claim 3, wherein when a slope of the first caloric profile is increasing, a slope of the second caloric profile decreases, and when a slope of the first caloric profile is decreasing a slope of the second caloric profile increases.

6. The in-ear stimulation system of claim 3, wherein when the first caloric profile cools one of the subject's ear canals, the second caloric profile heats the other of the subject's ear canals.

7. The in-ear stimulation system of claim 3, wherein the first and second caloric profiles are configured to maintain a vestibular stimulation of the subject for at least five minutes.

8. The in-ear stimulation system of claim 7, wherein the vestibular stimulation for at least five minutes is sufficient to be detected via functional imaging of a brain stem of the subject, heart rate modulation, and respiratory/breathing modulation.

9. The in-ear stimulation system of claim 3, wherein the first and second caloric profiles are configured to increase and/or decrease a temperature at the first and second conduits at a rate of about 20° C.-100° C., 30° C.-60° C. or 100° C. per minute or more.

10. The in-ear stimulation system of claim 1, wherein the conduit comprises an earpiece configured to be insertable into an ear canal of the subject, to receive the fluid from the fluid control system and to provide a thermal communication between the fluid and the ear canal of the subject.

11. The in-ear stimulation system of claim 10, wherein the earpiece comprises a three-dimensional printed earpiece having a fluid passageway therein.

12. The in-ear stimulation system of claim 10, wherein the earpiece comprises a thermally conductive housing that defines a chamber configured to receive the fluid therein.

13. The in-ear stimulation system of claim 10, wherein the earpiece comprises an outlet configured to release the fluid into the ear canal of the subject.

14. The in-ear stimulation system of claim 13, wherein the earpiece comprises a sealing member configured to enclose the fluid in the ear canal of the subject and an outlet configured to drain the fluid away from the ear canal of the subject.

15. The in-ear stimulation system of claim 1, wherein the fluid control unit further comprises a temperature sensor configured to detect a temperature of fluid in the conduit.

16. The in-ear stimulation system of claim 15, wherein the controller is configured to receive the temperature from the temperature sensor and to adjust the caloric profile of the fluid if the temperature from the temperature sensor is above or below a predetermined temperature range.

17. The in-ear stimulation system of claim 1, wherein the fluid control unit includes a Joule-Thomson expansion chamber.

18. The in-ear stimulation system of claim 17, wherein the Joule-Thomson expansion chamber includes a heating unit and/or a temperature sensor.

19. The in-ear stimulation system of claim 17, further comprising an earpiece in fluid communication with the Joule-Thomson expansion chamber.

20. A method for administering caloric stimulation to the ear canal of a subject, the method comprising:
(a) delivering a fluid to a conduit to calorically stimulate an ear canal of a subject; and
(b) controlling a flow of the fluid to the conduit and/or controlling a caloric profile of the fluid such that a temperature of the fluid changes through time when the fluid is being delivered to the ear canal via the conduit.

21. The method claim 20, wherein the caloric profile comprises a first caloric profile, the method comprising delivering a fluid to another ear canal of the according to a second caloric profile to provide dual ear caloric stimulation, wherein the first caloric profile is different than the second caloric profile.

22. The method of claim 21, wherein the first caloric profile is out-of-phase with the second caloric profile.

23. The method of claim 21, wherein when a slope of the first caloric profile is increasing, a slope of the second caloric profile decreases, and when a slope of the first caloric profile is decreasing a slope of the second caloric profile increases.

24. The method of claim 21, wherein when the first caloric profile cools one of the subject's ear canals, the second caloric profile heats the other of the subject's ear canals.

25. The method of claim 21, wherein the first and second caloric profiles are configured to maintain a vestibular stimulation of the subject for at least five minutes.

26. The method of claim 21, further comprising imaging a brain stem of the subject using functional imaging.

27. The method of claim 26, wherein functional imaging comprises at least one of Electroencephalography (EEG), Magnetoencephalography (MEG), functional Magnetic Resonance Imaging (fMRI), Positron Emission Tomography (PET) or Optical Imaging.

28. The method of claim 21, further comprising detecting cerebral blood flow using Doppler sonography.

29. The method of claim 21, wherein the first and second caloric profiles are configured to increase and/or decrease a temperature at the ear canal at a rate of about 20° C.-100° C., 30° C.-60° C. or 100° C. per minute or more.

30. The method of claim 20, wherein the conduit comprises an earpiece configured to be insertable into an ear canal of the subject, to receive the fluid therein and to provide a thermal communication between the fluid and the ear canal of the subject.

31. A stimulation device useful in a system for delivering caloric vestibular stimulation, comprising:
an earpiece configured to be insertable into an ear canal of a human subject, said earpiece having an external access portion; and
a first conduit in the earpiece, the first conduit having an inlet and an outlet, with both the inlet and the outlet positioned on the external access portion.

32. The device of claim 31, wherein the earpiece comprises a polymer material produced by three-dimensional printing.

33. The device of claim 31, further comprising a second conduit in the earpiece, said second conduit having an inlet and an outlet, with both the inlet and the outlet positioned on the external access portion.

34. The device of claim 31, wherein the earpiece comprises an internal cavity in fluid communication with the conduit.

35. The device of claim 31, wherein the earpiece external access portion comprises a heat exchange portion, and the conduit is positioned in the heat exchange portion.

36. The device of claim 31, wherein the conduit extends into the earpiece.
